(19) United States
(12) Patent Application Publication
(10) Pub. No.: US 2015/0305674 A1
(43) Pub. Date: Oct. 29, 2015

(54) Systems and Methods for Measuring Hydration in a Human Subject

(71) Applicant: Corewave LLC, Boulder, CO (US)

(72) Inventors: James Wright McPherson, Boulder, CO (US); James Steven Cunningham, Westminster, CO (US); Timothy G. Prodanovich, Boulder, CO (US); Peter Donald Gadsby, Broomfield, CO (US)

(73) Assignee: COREWAVE LLC, Boulder, CO (US)

(21) Appl. No.: 14/677,393

(22) Filed: Apr. 2, 2015

Related U.S. Application Data
(60) Provisional application No. 61/974,063, filed on Apr. 2, 2014.
(60) Provisional application No. 62/000,588, filed on May 20, 2014.
(60) Provisional application No. 62/007,671, filed on Jun. 4, 2014.

(51) Int. Cl. A61B 5/00; A61B 5/0205; A61B 5/026; A61B 5/0295; A61B 5/11

(52) U.S. Cl. CPC A61B 5/4875; A61B 5/0295; A61B 5/725; A61B 5/11; A61B 5/7257; A61B 5/7239; A61B 5/721; A61B 5/7203; A61B 5/0261; A61B 5/6806; A61B 5/6803; A61B 5/681; A61B 5/0205; A61B 5/002; A61B 5/0024; A61B 5/6898; A61B 5/7278; A61B 5/486; A61B 2562/0219; A61B 2562/0238

(57) ABSTRACT

A system for determining the hydration status of an individual utilizes a light sources and a photodetectors to extract a plethysmographic waveform from an individual and applies feature extraction to extract features from said plethysmographic waveform from which the hydration status of the individual can be inferred or calculated. The system of preferably housed in a wearable housing.

### RELATED APPLICATIONS

This application claims the benefit of the following co-pending U.S. Provisional Applications: U.S. Provisional No. 61/974,063, filed Apr. 2, 2014, U.S. Provisional No. 62/000,588, filed May 20, 2014 and U.S. Provisional No. 62/007,671, filed Jun. 4, 2014.

### FIELD OF THE INVENTION

This invention relates to the field of plethysmography, and, in particular, to a system and method of extracting features from a plethysmographic waveform to determine various characteristics of a human subject.

### BACKGROUND OF THE INVENTION

Water is vital in the life of all living things. It plays a central role in such functions as nutrient transport, waste removal, maintenance of cell volume, and thermal regulation. Assessment of hydration provides insight into basic biological processes and therefore is an important clinical parameter in determining health status and treatment of disease. A loss of only 2-3% of hydration by body weight is enough to lead to loss of physical performance, a loss of 6% can have serious repercussions, including heat exhaustion, and a loss of 8% and greater can lead to delirium, temporary deafness, cessation of urination and, at greater levels, death. Both dehydration and over hydration can have tragic consequences. Thus, maintaining a normal hydration state is a primary concern in all aspects of medicine. Although there are no quantitative measurements of hydration currently being used, qualitative parameters such as skin turgor, urine production, and the presence of tears are commonly used to determine a patient's hydration status. Although the laboratory setting lends itself to more quantitative assessments, accurate measurements of hydration are often difficult and time consuming. A more readily attainable, accurate, and quantitative measure of hydration would provide scientists with a powerful tool in studying health and disease.

The amount of water in the blood is perhaps the most relevant indicator of the overall hydration status of the body, as blood is delivered to almost every tissue in the body. Water is also contained in the cells of the body, referred to as intracellular fluid, which helps the cells maintain their shape, and facilitates the myriad processes that a living organism needs. Water is also present outside the cells, referred to as the extracellular reservoir. The remainder of the water in a human body is contained in the blood plasma. The water in the different compartments of the body's reservoirs is constantly shifting back and forth in response to changing physiological and environmental conditions, with blood generally being the vehicle of redistribution. Blood is also the gateway to many of the body's water loss mechanisms such as the kidney, breath, and sweating. Therefore, measuring the water content of the blood gives a good relative measurement of the hydration status of the individual.

It is well known in the art that the pumping of blood by the heart causes a pressure wave to form in the arteries and arterioles in the subcutaneous tissue, known as a plethysmographic waveform. This waveform can be detected by illuminating the skin with the light from a light-emitting diode (LED) and then measuring the amount of light either transmitted or reflected to a photodiode. When the plethysmographic waveform is detected in this manner, it is referred to as a photoplethysmogram.

Such plethysmographic waveforms are typically collected and used by a pulse oximetry device to detect oxygen saturation in the blood of a human subject. Pulse oximeters typically use LEDs transmitting light through the skin in two wavelengths, typically in the red at about 660 nm and in the infrared at about 940 nm. Waveforms at each wavelength can be collected using a transmissive method, where the light from each LED is passed through a translucent portion of the subject's body and detected by a photodiode on the other side. The absorption of light at these wavelengths differs significantly between blood loaded with oxygen and blood lacking oxygen. Oxygenated blood absorbs more infrared light and allows more red light to pass through, while deoxygenated blood allows more infrared light to pass through and absorbs more red light. The detected light from each wavelength can be compared to derive a plethysmographic waveform, an example of which is shown in FIG. 1.

### SUMMARY OF THE INVENTION

The present invention relates to using feature extraction from a plethysmographic waveform to determine the hydration status of an individual. The plethysmographic waveform contains information about a host of physiological properties, including the relative amount of water in the blood. This invention relates to a method for measuring the amount of water in the blood by algorithmic feature extraction of the plethysmographic waveform. A method of determining the subject's pulse rate is also detailed in this invention.

The sensing of a plethysmographic waveform is based on the principal that absorption of a specific wavelength of light energy in tissue varies with the amount of oxygenated blood in vessels such as arteries or arterioles. When the heart beats, the volume of blood increases and travels as a pressure wave through the circulatory system. When this pressure wave passes the sensor, more of the light energy is absorbed, and after the pressure wave has passed less light energy is absorbed. This time variant signal can be detected using a sensor consisting of a light source combined with a photodetector. Amplifying the signal gives an electrical representation of the plethysmographic waveform.

This invention relates to a sensor system capable of recording and analyzing the plethysmographic signal by means of either transmitted or reflected light energy, either separately or in combination. This system uses a single monochromatic light source, for example an LED, between the wavelengths of 400 nm and 2000 nm and a photo-detector, an example being a PIN photo diode. Light is projected into oxygenated tissue, and is transmitted or reflected (or both) into a photo-detector, producing a modulated electrical signal which is recorded as a photoplethysmographic waveform.

Another aspect this invention also relates to the use of a motion sensor, such as an accelerometer or gyroscope, to compensate for distortion in plethysmographic sensor signals induced by motion of the subject.

In accordance with the above, a system and algorithmic methods to measure the relative water content of the blood includes a sensor and a system capable of measuring, recording and performing analysis on a plethysmographic waveform such that relevant features can be extracted to measure the relative amount of water in the blood. This measurement allows for the determination of the relative hydration status of an individual, as well as their pulse rate.

In one embodiment of the invention, a system for the non-invasive measurement and recording of a photoplethysmographic waveform includes a light source with a narrow spectral bandwidth in combination with a photodetector capable of sensing light energy within the spectral bandwidth of the light source. The light energy is projected into tissue with oxygen carrying blood vessels, and either transmitted, reflected or both back through tissue to the photodetector. The light energy reaching the photodetector contains information of the complete plethysmographic waveform. A means for analyzing the waveform to extract biometric information such as hydration status and heart rate is in communication with the photodetector for receiving information about the plethysmographic waveform.

In a second embodiment of the invention, the system is also provided with a motion sensor, such as an accelerometer or gyroscope, that measures the relative motion of the sensing environment, which tends to distort the plethysmographic waveform. This motion information can subsequently be subtracted from the distorted plethysmographic waveform, thereby allowing for the recovery of the true plethysmographic waveform from the distorted waveform.

### BRIEF DESCRIPTION OF THE DRAWINGS

FIG. 1, shows an example of raw plethysmographic data with a small amount of electrical noise and baseline wander.

FIG. 2, shows the plethysmographic waveform in FIG. 1 after it has been processed to remove noise, baseline wander and DC offset, and has been inverted for ease in processing.

FIG. 3, shows the plethysmographic waveform in FIG. 2 with its corresponding RMS value of 0.0012 Vrms.

FIG. 4, shows the frequency spectrum of the plethysmographic waveform in FIG. 2 derived using an FFT algorithm.

FIG. 5, shows an illustration of the concept of using the 1^{st} derivative to determine the slope of the line of a waveform.

FIG. 6, shows an example of the 1^{st} derivative derived from a plethysmographic waveform. The solid line is the derivative and the dashed line is the waveform it was derived from.

FIG. 7, shows a plethysmographic waveform (a) with its associated first (b) and second (c) derivatives.

FIG. 8, shows an example of a plethysmographic waveform that has the salient features for this invention called out: systolic peak-to-peak, diastolic peak-to-peak, the dicrotic notch, and the notch minimum.

FIG. 9, shows a flowchart of the algorithm for converting the plethysmographic data from its raw form to a number representative of hydration status.

FIG. 10, is a diagram of a transmissive photoplethysmographic sensor. The sensor consists of a light source, shown as LED in the diagram, and a photo-sensor, shown in the figure as a photodiode. The light energy passes through the tissue and the photo-sensor detects light that is not absorbed. This is similar to how traditional pulse oximetry is accomplished. The difference with the current invention is that a single wavelength of light is used instead of two as with traditional pulse oximetry.

FIG. 11, is a diagram of a reflective photoplethysmographic sensor. The sensor consists of a light source, shown as a single LED in the diagram, and a photo sensor shown in the figure as a photodiode. The light energy passes through the tissue is reflected off the surface of bone, and the photo-sensor detects light that is not absorbed. A single wavelength of light is used instead of two as with traditional pulse oximetry.

FIG. 12 shows an embodiment of the invention utilizing both a transmissive and reflective photoplethysmographic sensor. The sensor uses a single wavelength of light and additional information can be extracted from the differences in the signals obtained.

FIG. 13, is a diagram of an example of a self-contained transmissive and/or reflective sensor mounted on a finger. This self-contained photoplethysmographic sensor can communicate wirelessly with a device such as a smartphone or tablet to analyze and display information.

FIG. 14, shows an embodiment of a transmissive plethysmographic sensor integrated into a glove, with the inclusion of a microcontroller or other data processor along with a wireless communication capability.

FIG. 15 shows an embodiment of a wrist worn reflective plethysmographic sensor design with a self-contained microprocessor, wireless communication capability, and display.

FIG. 16 shows an example of a plethysmographic waveform with a large motion artifact generated when the finger that was being measured was moved suddenly.

FIG. 17 shows and example of the motion signal as measured by a motion sensor such as an accelerometer or gyroscope.

FIG. 18 shows the recovered plethysmographic waveform after the sensed motion waveform has been scaled to an appropriate level and subtracted from the dataset to reconstruct the true plethysmographic signal.

FIG. 19 is an example of a self-contained transmissive and/or reflective sensor mounted on a finger with and accelerometer attached to the sensor. This puts the motion sensor in the same inertial reference point as the plethysmographic sensor, allowing for reconstruction of the physiological information.

FIG. 20 shows a flow chart of the basic algorithmic process for reconstructing a plethysmographic waveform that has been distorted by motion.

FIG. 21 is a high level block diagram showing the hardware components of the system.

FIG. 22 is a graph showing an example of hydration status over time, where the hydration status has been derived using the FFT embodiment of the invention.

### DETAILED DESCRIPTION

This invention relates to using feature extraction from a plethysmographic waveform to determine the hydration status of an individual. The plethysmographic wave is the pressure wave present in the arteries and arterioles of the body that is generated by the beating of the heart. This waveform contains information about a host of physiological properties, including the relative amount of water in the blood. It has been found that an inference can be made as to water content of the entire body based on the water content in the blood plasma.

This invention comprises the use of one or more of six different algorithmic methods to extract the salient features from the plethysmographic waveform for the measurement of the amount of water in the blood. These methods are:
Fourier Transform
Root Mean Square (RMS)
Derivative (rate of change)
Systolic Peak to Peak (SP)
Crest Factor:

$$C = \frac{SP}{RMS}$$

Peak to Average Power (PAPR) ratio: PAPR=C²

Each of these methods is a mathematical calculation which can be used to analyze the salient features of the plethysmographic waveform to determine the overall hydration status of the human body. In each case, these mathematical operations provide a number from which an inference can be made as to the amount of water in the blood.

Before the plethysmographic waveform can be analyzed, the raw data needs to be processed to remove noise and baseline wander. The data may also be inverted to make the waveform conform to the normal plethysmographic standard, but this for convenience and is not an absolute requirement for the processing of the data. The plethysmographic waveform can be processed in the inverted or non-inverted form.

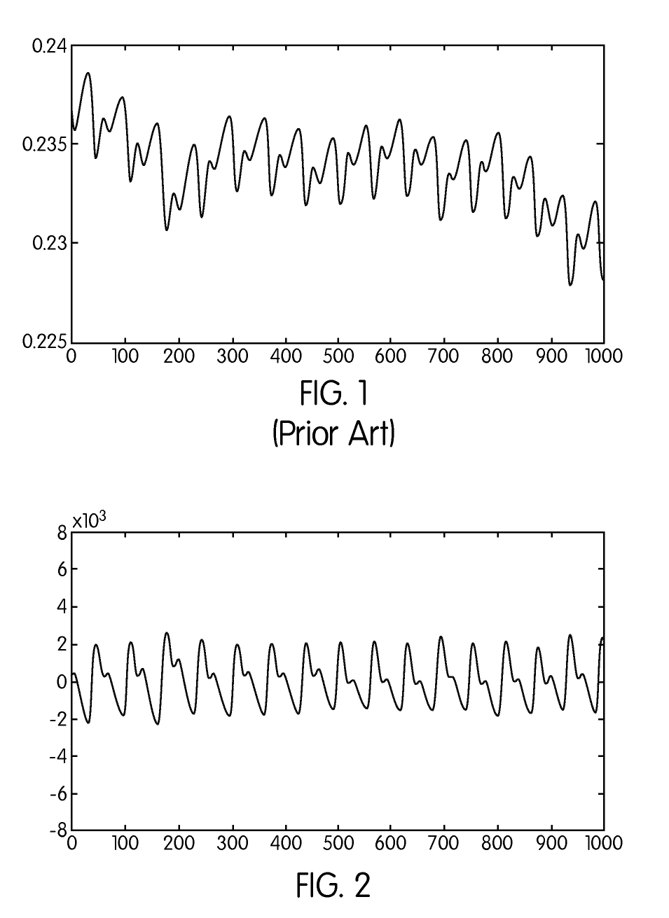

Noise in the waveform can occur due to either electrical or mechanical factors, such as 60 Hz power line noise or a mechanical vibration induced into the patient. Baseline wander is the slowly changing DC bias variation caused by subtle changes in the DC bias of the sensor amplifiers, which causes a DC offset in the waveform. FIG. 1 shows a raw un-inverted plethysmographic waveform with a small amount of power line noise and baseline wander.

Because the frequencies of interest in the plethysmographic waveform related to this invention are below 15 Hz, proper selection of sample frequency and low pass filtering of the signal are usually sufficient to remove the electrical noise from the signal. Baseline wander is removed by subtracting the long term running average of the signal. FIG. 2 shows the signal of FIG. 1 after it has been inverted, filtered, and had the baseline wander removed. The plethysmographic waveform in FIG. 2 is an example of a plethysmographic waveform obtained from measuring the absorption of infrared light transmitted through oxygenated tissue. The peak of the waveform corresponds to the maximum absorption of the IR light when the blood vessels, such as arterioles, are pulsing at their maximum dilation, and the lowest part of the waveform is the point between heart beats where there is the minimum dilation of the vessels and correspondingly less absorption of the light.

Once the signal has been processed such that it is symmetrically periodic around zero, with all DC bias removed, the waveform is ready for feature extraction to determine the amount of water in the blood.

Those skilled in the art will understand that practical methods of measuring and recording a biometric waveform, such as a plethysmogram, involve analog to digital sampling of the waveform which has the effect of breaking the waveform into discrete samples representative of the original, inherently analog waveform. This discrete data is now ready for analysis by a microcontroller or a microprocessor.

### Fourier Transform

The first method of feature extraction related to this algorithm is to obtain a frequency spectrum of the waveform through the use of Fourier transform analysis. A Fourier transform converts time or space information to frequency. Of particular usefulness, is the method known as the fast Fourier transform (FFT), as it can be used to process a discrete dataset in a computationally fast and efficient manner. This is the preferred embodiment of the invention. Equation 1 shows the mathematical relation between the Fourier transform and the function f(x) that represents the waveform.

f(ξ)=∫_{-∞}^{∞}f(χ)e^{-2πixt}dx, for any real number ζ (1)

An FFT is an algorithm to compute the discrete Fourier transform (DFT) and it's inverse. The DFT is defined by the formula shown in Equation 2.

$${X}_{k} = {\sum }_{n = 0}^{N - 1} {x}_{n} {e}^{- i2 π k \frac{n}{N}} \\ k = 0 , … , N - 1. \qquad ( 2 )$$

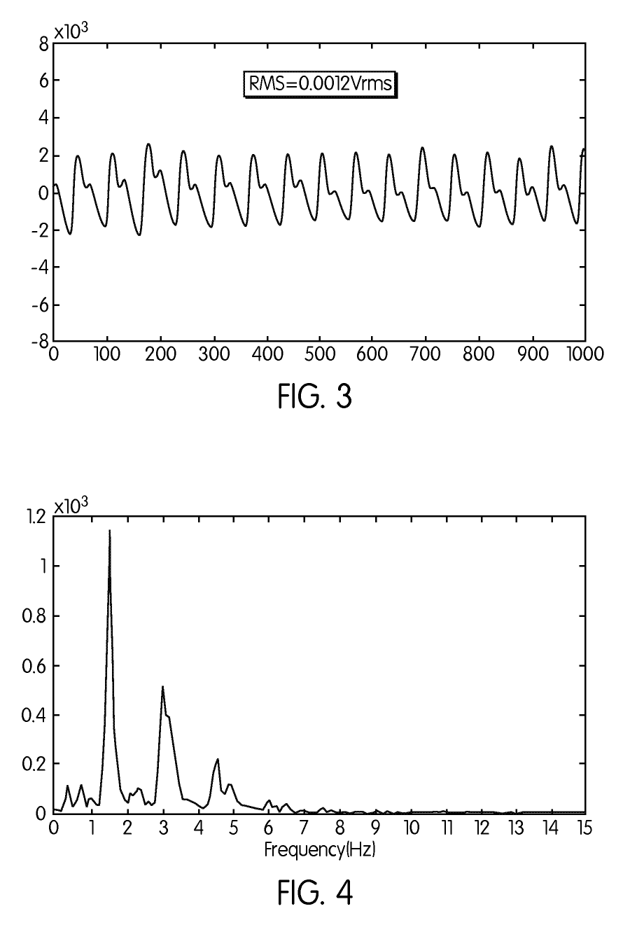

An FFT computes the DFT and produces the same result as evaluating the DFT definition directly; the most important difference is that an FFT is much faster. The most common method is the Cooley-Tukey FFT algorithm, but others include the Prime-factor FFT algorithm, Bruun's FFT algorithm, Rader's FFT algorithm, and Bluestein's FFT algorithm. Algorithmically, and as related to this invention, the magnitude of the maxima of the frequency spectrum returned by a Fourier transform, specifically the first (primary) harmonic of a plethysmographic waveform, is inversely proportional to the hydration status of the individual the waveform is taken from. FIG. 4 shows an example of an FFT obtained from the plethysmograph of FIG. 3. The first harmonic can be observed as the large spike in the waveform.

Additionally, the spectrum produced by the Fourier transform can be used to measure the pulse rate of an individual. The first harmonic of the spectrum is the fundamental frequency and therefore represents the heart rate. To determine heart rate, the first harmonic maxima can be multiplied by sixty with the resulting number being the heart rate in beats per minute, as shown in Equation 3.

HR=f1st(60) (3)

Root Mean Square

The first step in the feature extraction related to this algorithm is to determine the Root Mean Square (RMS) of the plethysmographic waveform. In mathematics, the root mean square, also known as the quadratic mean, is a statistical measure of the magnitude of a varying quantity. The RMS of a waveform can be calculated for either a continuous or a discrete signal. Equation 4 shows the mathematical relation for continuous case where the waveform function RMS is between times T1 and T2, and Equation 5 shows it for the discrete case with n set of values.

$$\begin{matrix}{f}_{rms} = \sqrt{\frac{1}{{T}_{2} - {T}_{1}} {\int }_{{T}_{1}}^{{T}_{2}} {[ f ( t ) ]}^{2} d t} , & ( 4 ) \\ {x}_{rms} = \sqrt{\frac{1}{n} ( {x}_{1}^{2} + {x}_{2}^{2} + … + {x}_{n}^{2} )} . & ( 5 )\end{matrix}$$

In either case, the result returned is a real number that can be used to analyze a waveform or set of data. Algorithmically, as related to this invention, the RMS value of the plethysmographic waveform is inversely proportional to the hydration status of the individual from which the waveform is taken. FIG. 3 shows an example plethysmographic waveform, and its corresponding RMS value. The plethysmographic waveform can be processed in the inverted or non-inverted form.

### Derivative

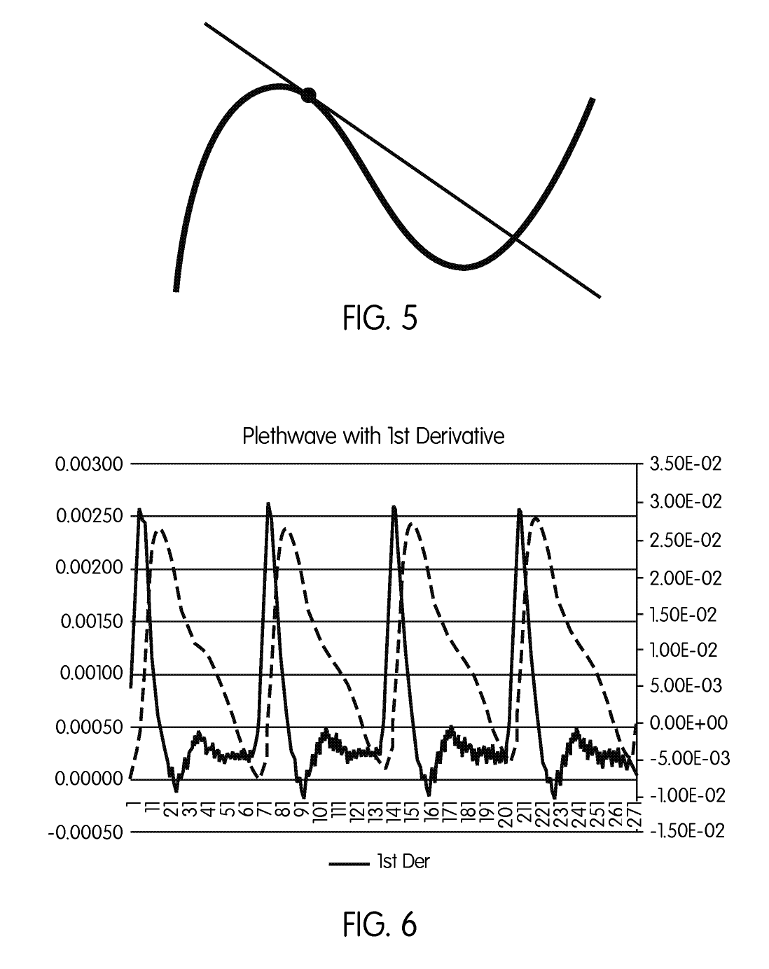

The derivative of an equation or a waveform gives the slope of the line that is tangential to the function or waveform at that specific point. FIG. 5 shows an illustration of this concept. Equation 6 below shows one of the formal definitions of the derivative of a function.

$${f}^{′} ( a ) = {lim}_{h -> 0} \frac{f ( a + h ) - f ( a )}{h} \qquad ( 6 )$$

Algorithmically, as related to this invention, the magnitude of the derivative of the plethysmographic waveform, meaning the slope of the line at any given point in the waveform, is inversely proportional to the hydration status of the individual from which the waveform is taken. The maximum derivative values can be used alone or in combination with the other maximum derivative values to assess hydration.

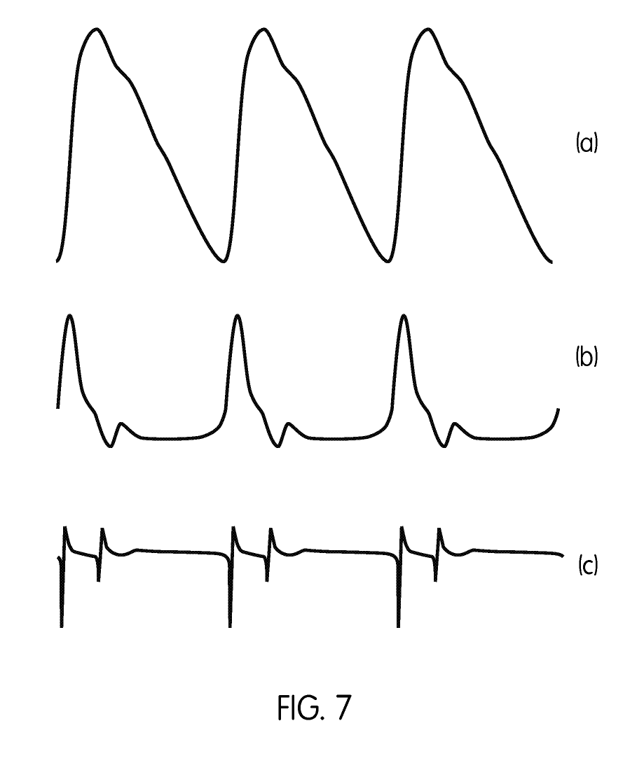

FIG. 6 shows an example of the first derivative obtained from a plethysmographic waveform. The first derivative (solid line) is a measure of the rate of change of a waveform, and the second derivative (dotted line) is a measure of the rate of change and is also useful for feature extraction along with being useful for the timing of the waveform. FIG. 7 shows an example of a plethysmographic waveform (a) and its associated 1^{st} (b) and 2^{nd} (c) derivatives.

### Systolic Peak to Peak

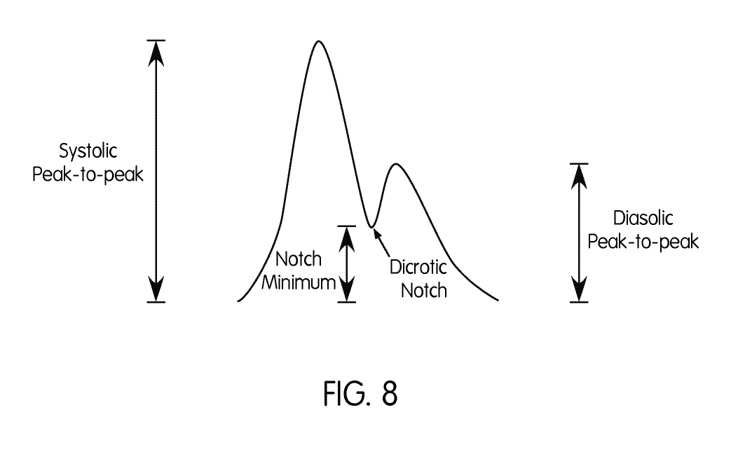

FIG. 8 shows a representation of a plethysmographic waveform and its main features. The systolic peak-to-peak value is the maximum amplitude of the waveform relative to prior minima that occurs just before the heart contracts. The magnitude of the systolic peak-to-peak measurement is inversely proportional to the hydration status of the individual.

### Crest Factor

The crest factor is the ratio of the systolic peak amplitude to the RMS of the waveform, as shown in Equation 7. The magnitude of the crest factor measurement is inversely proportional to the hydration status of the individual.

$$C = \frac{SP}{RMS} \qquad ( 7 )$$

### Peak to Average Power Ratio

The peak to average power (PAPR) ratio is the square of the crest factor and is shown in Equation 8. The magnitude of the PAPR measurement is inversely proportional to the hydration status of the individual.

A single wavelength optical biometric sensor system such as described and used to measure a plethysmographic waveform is prone to noise and signal distortion due to physical movement of the subject. The time variance and frequencies of such movement are on the same order as that of biological signals, with the plethysmographic pressure wave being a preferred example. Therefore, in addition to the sensing and recording of a plethysmographic waveform, the present invention comprises a motion sensor in direct proximity of the apparatus which senses the transmissive or reflective light. The sensed motion information can be used to compensate for motion distortion of the plethysmographic waveform, allowing for the recovery of the biometric plethysmographic signal.

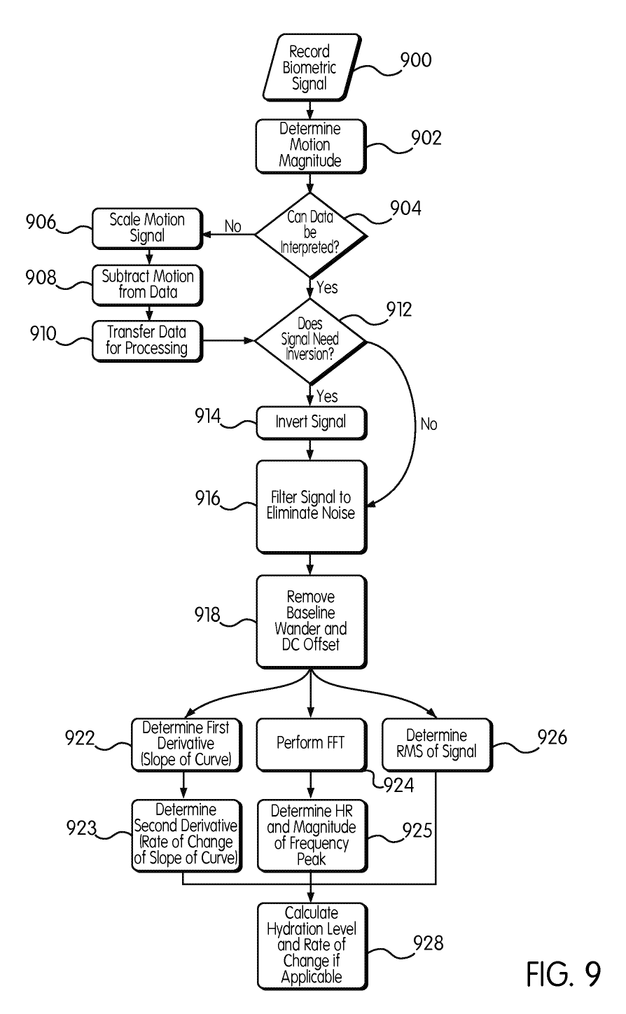

FIG. 9 shows a flow chart of the preprocessing steps to analyze a plethysmographic waveform and three of the possible algorithmic methods. At 900, the plethysmographic waveform is recorded. As those skilled in the art will appreciate, the signals received from the sensor are preprocessed, including an analog to digital conversion, to a format that is amiable to analysis, wherein the algorithm uses feature extraction of the plethysmographic waveform to measure the hydration status of an individual. At 902, it is determined if there is distortion in the plethysmographic waveform due to motion and at 904, it is determined if the plethysmographic waveform can be interpreted or if correction for the motion must be applied. If correction is required, the motion signal is scaled at 906. And, at 908, the motion signal is subtracted from the plethysmographic waveform. At 910 the data is made available for further processing. At 912-918, the signal is manipulated and filtered to obtain a normalized waveform ready for analysis. At 912, it is determined if the signal requires inversion, and, if so, at 914, the signal is inverted. At 916, the signal is filtered to remove noise and at 918, the signal is normalized to remove baseline wander and DC offset.

One of three preferred methods of extracting the hydration information from the plethysmographic waveform is then utilized. It should be noted that not all three algorithms will be programmed into the system. This portion of FIG. 9 merely illustrates that any one of three preferred methods could be utilized to implement the invention.

At 922, the derivative method, discussed above, is utilized. At 922, the first derivative of the plethysmographic waveform is derived, showing the slope of the curve. At 923, the second derivative is calculated, showing the rate of change of the slope curve calculated in 922. The rate of change of the derivative of the plethysmographic waveform, meaning the second derivative of the line at any given point in the waveform, is inversely proportional to the hydration status of the individual from which the waveform has been obtained.

At 924, the FFT method is used. The FFT method is the preferred embodiment of the invention. At 924, the FFT of the plethysmographic waveform is calculated and, at 925, the pulse rate is determined the based on the frequency of the first harmonic of the frequency spectrum, obtained using a Fourier Transform or another algorithm.

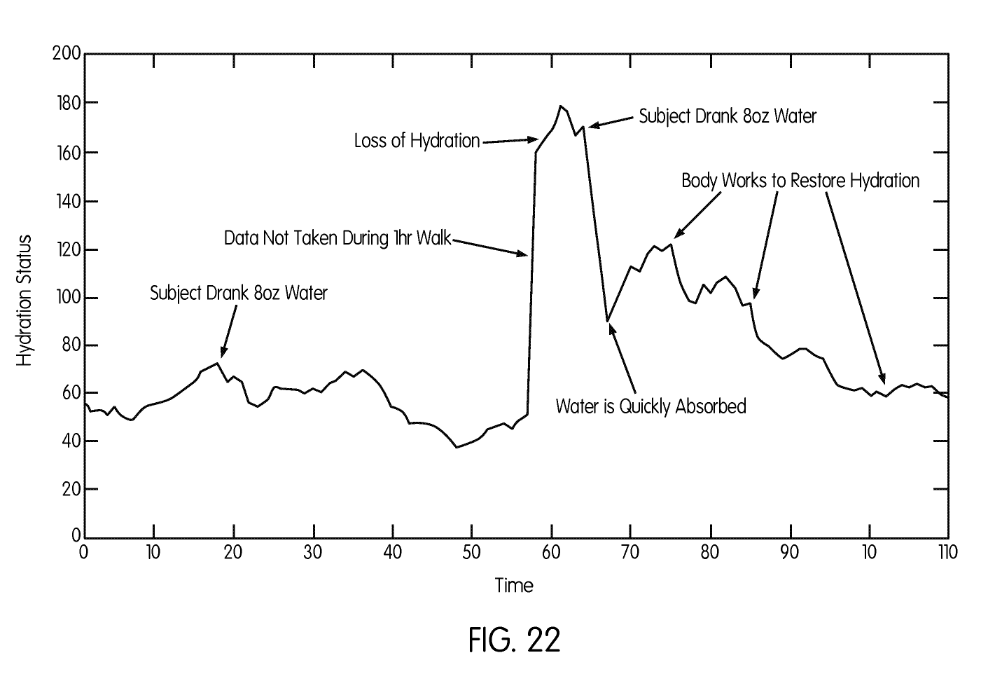

The hydration status of the individual is based on the magnitude of the peak frequency (i.e., the primary harmonic). Generally, the lower the magnitude, the more likely it is that the subject is de-hydrated. The exact magnitude to distinguish between a properly-hydrated state and a state of dehydration varies for each individual. Therefore, the system of the present invention will need to be calibrated on an individual basis. The FFT method for determining hydration status has been demonstrated through hydration testing and a small clinical study. The magnitude of the FFT primary harmonic correlates to the amount of blood volume, of which water is the largest component. FIG. 22 shows an example of real hydration data gathered via this method, with the x-axis showing time, and the y-axis showing hydration status as a function of the magnitude of the primary harmonic.

At 924, the last preferred method of interpreting the plethysmographic waveform is utilized. The RMS of the plethysmographic waveform is calculated. Generally, the RMS value of the plethysmographic waveform is inversely proportional to the hydration status of the individual. As with the other methods, the system of the present invention will need to be calibrated for each individual.

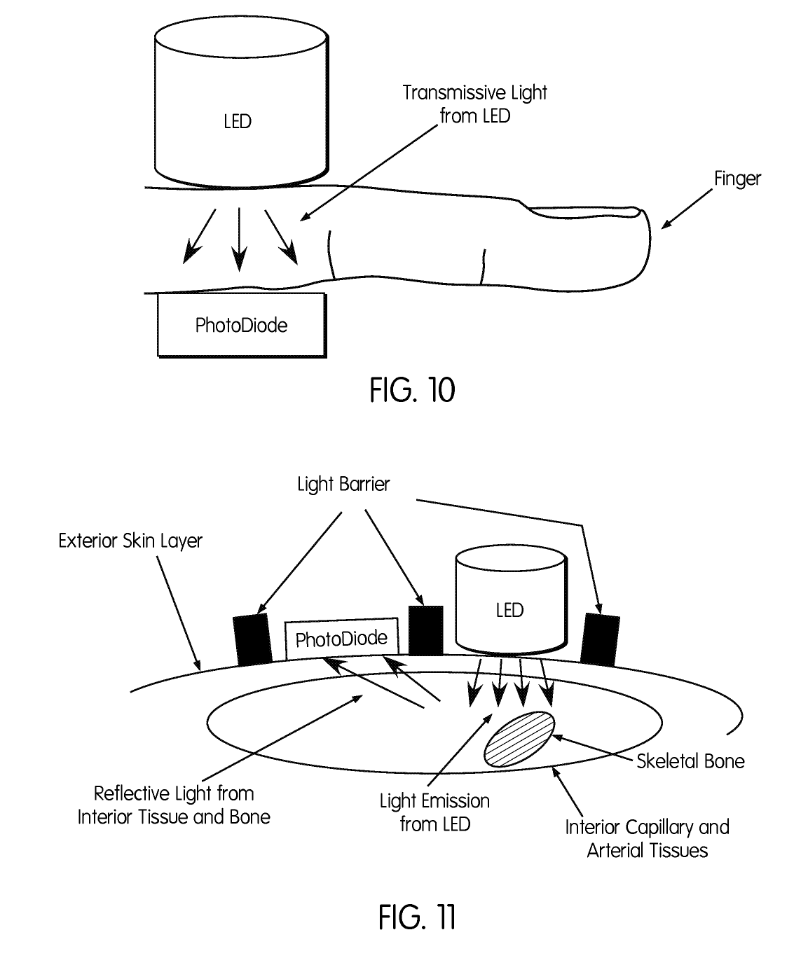

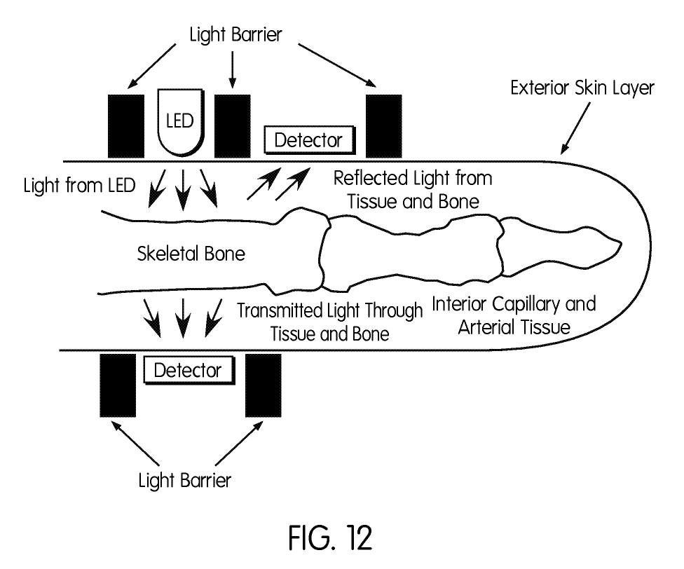

FIG. 10 shows a representation of an embodiment of the present invention using transmitted light passing through tissue and being detected by a photo-sensor, while FIG. 11 shows a representation of reflected light being detected by a photo-sensor. In addition to using either a transmissive or reflective plethysmographic sensor in a wearable device, a combination of sensors can be incorporated. FIG. 12 shows an example of a sensor where both a transmissive and reflective measurements methods are used. This configuration gives an additional layer of physiological information, allowing for differential measurements and cancellation of non-relevant information.

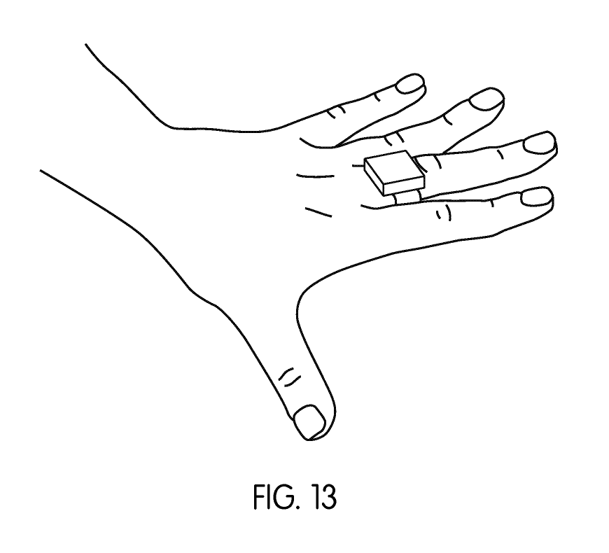

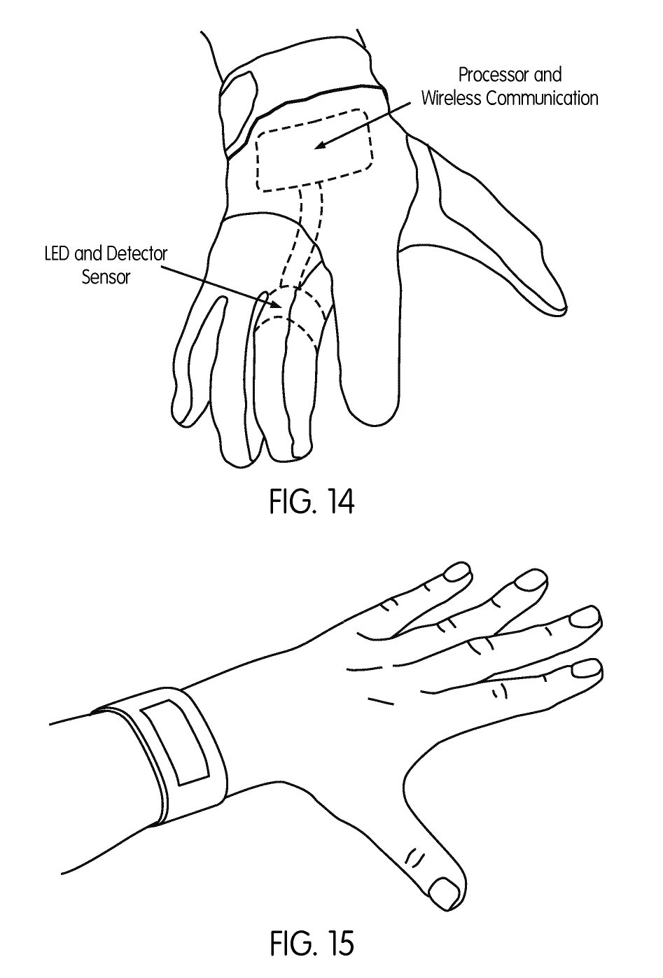

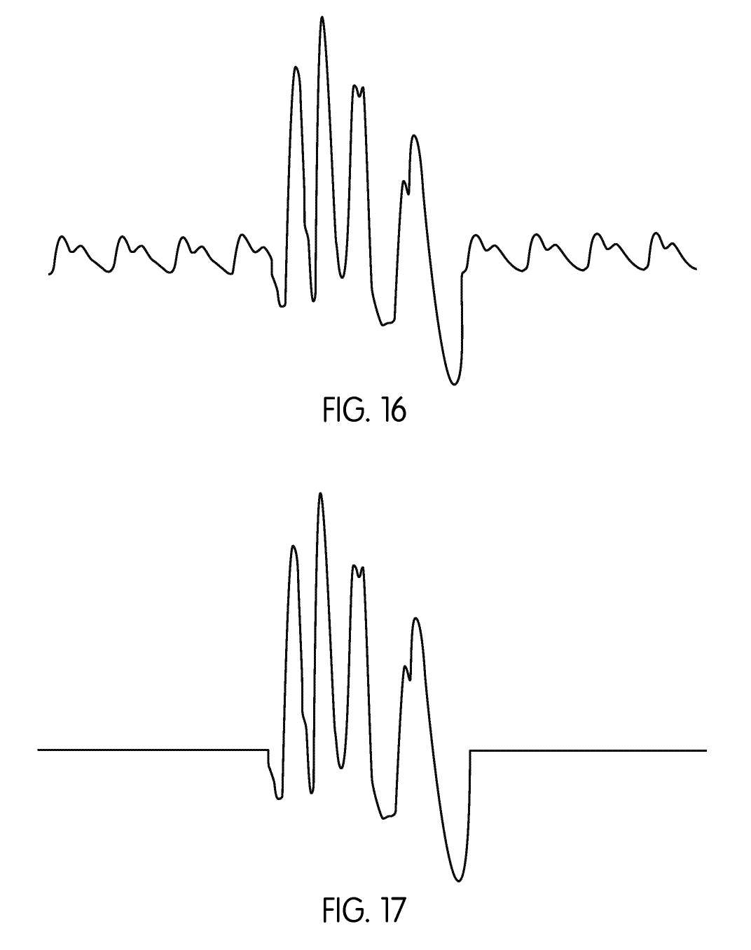

FIGS. 13, 14 and 15 show various examples of wearable versions of the plethysmographic sensors of the present invention. These sensors are may be integrated into various wearable articles and would likely also be equipped with the motion sensing feature described above, with the motion sensor mounted at the same inertial reference point as the sensor for the plethysmographic waveform. FIG. 13 shows a device incorporated into a ring, while FIG. 14 shows a glove and FIG. 15 a wristband. One of skill in the art can imagine that such sensors could be incorporated into any wearable item, including, for example, glasses, socks, shoes, undergarments, hats, etc.

Regarding the processing of the plethysmographic waveform to remove motion artifacts, FIG. 16 shows an example of a plethysmographic waveform with a motion artifact induced by the movement of a finger. This induced distortion of the plethysmographic waveform is of a much higher magnitude than the desired biometric signal, thereby inducing so much distortion that it would appear all useful information would be lost.

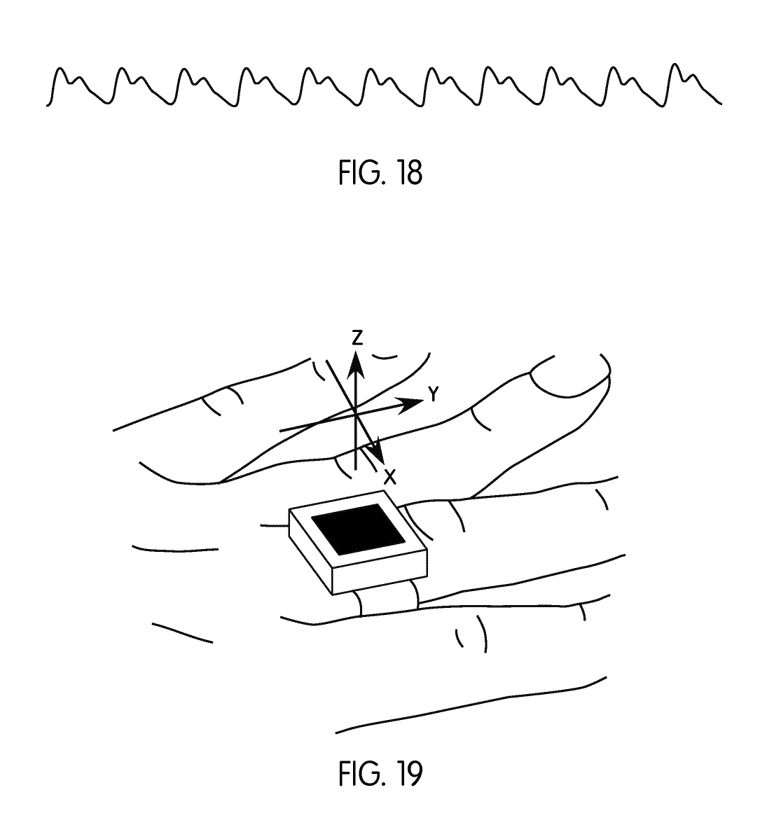

However, FIG. 17 shows the same motion artifact as sensed by a motion sensor, such as an accelerometer or a gyroscope. This information from the motion sensor can be re-scaled to the appropriate level and then subtracted from the stored biometric data at the same time index as when the disturbance occurred. The subtraction of the scaled motion sensor data allows for the reconstruction of the original plethysmographic waveform, as shown in FIG. 18. FIG. 19 shows an example of a plethysmographic sensor with an accelerometer mounted in the same inertial frame of reference as the sensor.

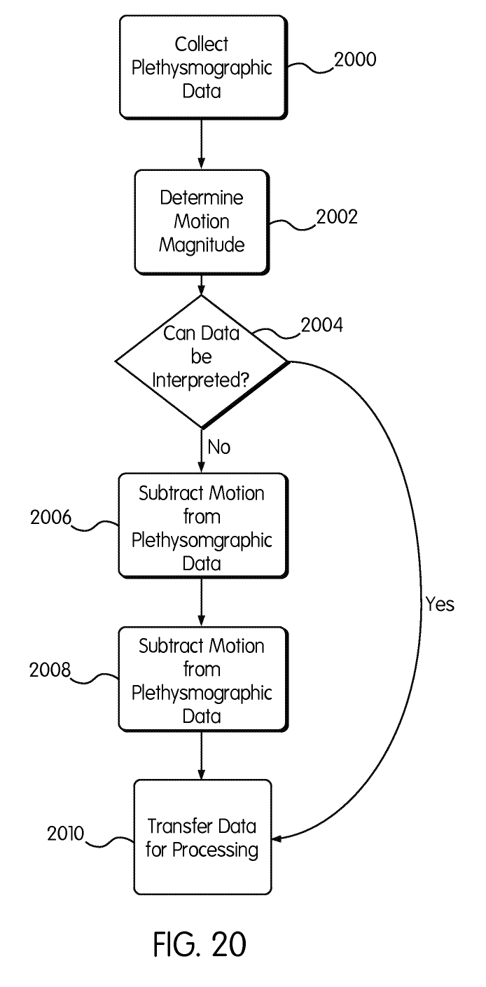

FIG. 20 shows a flow chart of the algorithmic methodology behind removing distortion induced by motion. At 2000, the plethysmographic waveform and motion waveform are collected. At 2002, the magnitude of the magnitude of the motion waveform is examined and at 2004 it is determined if compensation for the motion to normalize the plethysmographic waveform is necessary. If compensation is necessary, at 2006 the motion waveform is scaled to the same scale as the plethysmographic waveform and, at 2008, the motion waveform is subtracted from the plethysmographic waveform. At 210, the corrected plethysmographic waveform is ready for feature extraction. If no compensation for motion is necessary in 2004, processing proceeds directly to 2010 for feature extraction.

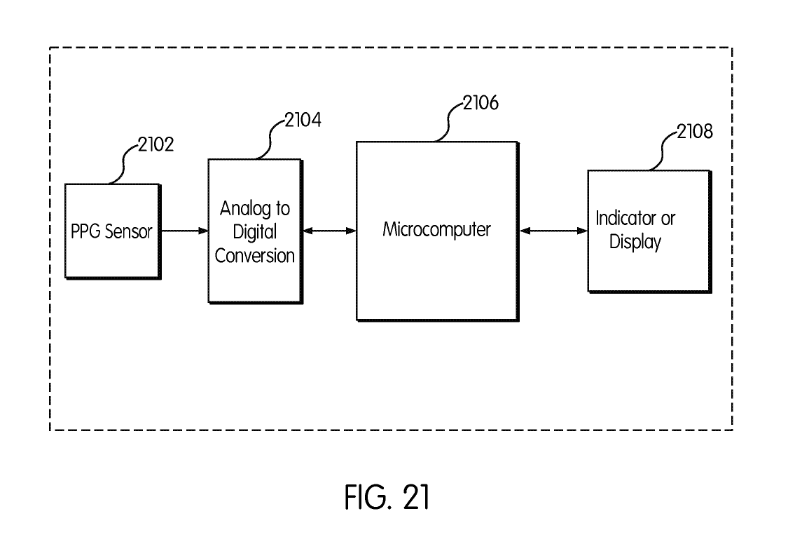

A high level block diagram of the hardware components of the invention is shown in FIG. 21. Component 2102 is the sensor for the photoplethysmographic (PPG) waveform and consists of one or more light sources, preferably LEDs, emitting light in a narrow wavelength band and one or more light detectors, preferably photodiodes. Raw data from the photodiodes (and motion sensor, if present), is sent to A-to-D converter 2104 where a digital signal is produced. This digital signal is sent to microcomputer 2106, running software for performing the interpretation functions already discussed. Results of the interpretation are displayed by some form of indicator or display 2108.

The foregoing invention has been explained in terms of function and purpose, and, as would be realized by skill in the art, may be implemented using many methods. For example, the portion 2106 of the invention that manipulates the plethysmographic waveform and the motion waveform may be implemented as software running in a general purpose computer, as hard circuitry, as a microprocessor running firmware, as a functionally programmed ASIC, or by using any other method known in the art. In any case, the sensor portion of the invention 2102, namely the LED(s), photodetector(s) and motion detector need to be configured to be in communication with the analysis components of the implementation. This can be accomplished via A-to-D converter 2104, which transforms the raw analog signal produced by the photodiodes and motion detector into a digital signal for processing by microcomputer 2106.

In one implementation of the invention, the device is entirely self-contained, including a means for communicating the results of the hydration analysis to the subject. Other possible implementations of the invention could communicate raw sensor data, a digitized version of the raw sensor data, or completely analyzed results to another device, for example, a mobile device such as a tablet or smartphone via, for example, WiFi or Bluetooth. The mobile device could provide the mathematical analysis of the raw sensor data or could be used merely to display results.

In addition, there will need to be a method of communicating the hydration status of the subject. This could be a simple as an LED indicating a dehydrated status, or more complicated, such as an LCD readout providing a numerical indication of the hydration status, or through the use of a “fuel gauge”, where a graphical indicator gives an indication of hydration status. In cases where the hydration status is displayed by a mobile computing device, more complicated feedback could be provided, for example, a graph of the subject's hydration status throughout the day or over longer periods. The system may also provide suggestions as to how the subject may improve his or her hydration status.

We claim:

1. A system for determining the hydration status of an individual comprising:
a. one or more light sources;
b. one or more photodetectors; and
c. software, running on a computing platform, said software performing the functions of:
i. receiving data from said one or more photodetectors, said received data containing a plethysmographic waveform obtained from said individual;
ii. performing a feature extraction on said plethysmographic waveform to extract features indicative of the overall hydration status of said individual; and
iii. interpreting said extracted features and providing feedback to said individual regarding said hydration status.

2. The system of claim 1 wherein said software performs the further functions of filtering and normalizing said plethysmographic waveform prior to performing said feature extracting step.

3. The system of claim 2 further comprising:
a. a motion sensor;
b. wherein said software performs the further functions of:
i. receiving data from said motion sensor; and
ii. manipulating said plethysmographic waveform to correct for motions of said individual based on said data received from said motion detector.

4. The system of claim 1 wherein said computing platform is separate from said light sources and photodetectors, further comprising a means of communicating data between said photodetectors and said computing platform.

5. The system of claim 4 wherein said means for communicating is wireless.

6. The system of claim 5 wherein said means for communicating is BlueTooth or WiFi.

7. The system of claim 4 wherein said computing platform is a mobile computing device.

8. The system of claim 7 wherein said mobile computing device is a smartphone or a tablet.

9. The system of claim 1 wherein said step of feature extraction comprises:
a. obtaining a frequency spectrum of said plethysmographic waveform; and
b. interpreting said hydration status of said individual as a function of the magnitude of the primary harmonic of said frequency spectrum.

10. The system of claim 10 wherein said frequency spectrum is obtained by using a Fast Fourier Transform algorithm.

11. The system of claim 9 wherein said software performs the further function of extracting pulse rate information from said frequency spectrum, said pulse rate being interpreted as a function of the frequency of the primary harmonic of said frequency spectrum.

12. The system of claim 1 wherein said step of feature extraction comprises:
a. obtaining root mean square value of said plethysmographic waveform; and
b. interpreting said hydration status of said individual as a function of said mot mean square value.

13. The system of claim 1 wherein said step of feature extraction comprises:
a. calculating the first derivative of said plethysmographic waveform; and
b. interpreting said hydration status of said individual as a function of the peak values of said first derivative.

14. The system of claim 1 wherein said step of feature extraction comprises:
a. measuring the peak-to-peak magnitude of the systolic portion of said plethysmographic waveform; and
b. interpreting said hydration status of said individual as a function of said peak-to-peak magnitude.

15. The system of claim 1 wherein said step of feature extraction comprises:
a. measuring the systolic peak amplitude of said plethysmographic waveform;
b. measuring the RMS value of said plethysmographic waveform; and
c. interpreting said hydration status of said individual as a function the ratio of said systolic peak amplitude to said RMS value.

16. The system of claim 2 wherein said step of filtering and normalizing said plethysmographic waveform includes the step of inverting said plethysmographic waveform prior to the step of feature extraction.

17. The system of claim 2 wherein said step of filtering and normalizing said plethysmographic waveform includes the step of filtering said plethysmographic waveform to remove electrical noise and mechanical vibration.

18. The system of claim 17 wherein said filtering is accomplished using low pass filtering, high pass filtering, band pass filtering, and running averaging of said plethysmographic waveform.

19. The system of claim 2 wherein said step of filtering and normalizing said plethysmographic waveform includes the step of processing said plethysmographic waveform to remove baseline wander and DC offset by subtracting the long term running average of the plethysmographic waveform from the plethysmographic waveform.

20. The system of claim 1 wherein said light sources are LEDs supplying light having a wavelength in the range of 400 nm to 2000 nm.

21. The system of claim 1 wherein said light sources and said photodetectors are located on opposite sides of a body part of said individual, such that light detected by said photodetectors has been transmitted through said body part.

22. The system of claim 1 wherein said light sources and said photodetectors are located on the same side of a body part of said individual, such that light detected by said photodetectors has been reflected from by said body part.

23. The system of claim 1 wherein said photodetectors are located on the same side of a body part of said individual, and on opposite sides of the body part of an individual with respect to said light sources, such that light detected by said photodetectors has been both transmitted through and reflected by said body part.

24. The system of claim 1 wherein said system is housed in a housing wearable by said individual.

25. The system of claim 24 wherein said housing includes gloves, clothing, expandable bandages, sweat bands, bicycle helmets, sportswear, cell phone arm bands, wrist bands, and a finger ring.

26. The system of claim 3 wherein said motion sensor is selected form a group comprising one or more accelerometers and a gyroscope.
